(19) United States
(12) Patent Application Publication
(10) Pub. No.: US 2023/0218693 A1
(43) Pub. Date: Jul. 13, 2023

(54) HEMP SPROUT PRODUCTS AND METHODS FOR PRODUCING SAME

(71) Applicant: CReDO Science, LLC, Austin, TX (US)

(72) Inventor: Ethan Russo, Vashon, WA (US)

(21) Appl. No.: 18/000,918

(22) PCT Filed: Jun. 11, 2021
(86) PCT No.: PCT/US2021/037082

§ 371 Date: Dec. 6, 2022

Related U.S. Application Data
(60) Provisional application No. 63/038,404, filed on Jun. 12, 2020.

(51) Int. Cl. A61K 36/185; A61K 31/352; A61K 31/122; A61K 31/05; A61K 31/015; A61K 9/00; A61K 31/09; A61P 29/00; A23L 33/105; A23L 33/00

(52) U.S. Cl. CPC A61K 36/185; A61K 31/352; A61K 31/122; A61K 31/05; A61K 31/015; A61K 9/0053; A61K 31/09; A61P 29/00; A23L 33/105; A23L 33/40

(57) ABSTRACT

The present invention relates to hemp products, such as sprouted hemp seed products, and methods for producing the same. The methods preserve the nutritional values of hemp seeds prior to processing.

### CLAIM OF PRIORITY UNDER 35 U.S.C. § 119

The present Application for Patent claims priority to Provisional Application No. 63/038,404 entitled “HEMP SPROUT PRODUCTS AND METHODS FOR PRODUCING SAME” filed Jun. 12, 2020 and assigned to the assignee hereof and hereby expressly incorporated by reference herein.

### BACKGROUND

### Field

The present invention relates to hemp products, such as sprouted hemp seed products, and methods for producing the same.

### Background

Hemp seeds are considered to be a nutritionally complete food and powerful anti-inflammatory for numerous reasons. For one, hemp seeds contain all essential amino acids. Hemp seeds are comprised of 35% protein, as digestible edestin and 35% oil. Also, they are rich in essential fatty acids (EFA) in 3:1 ω6:ω3 ratio: 56% linoleic acid (LA, ω-6), 22% linolenic acid (LNA, ω-3), 4% gamma-linolenic acid (GLA, ω-6) (Callaway 2004).

### SUMMARY

Some embodiments of the invention relate to a method of making a nutritional product. In some embodiments, the method can include providing a hemp variety with a zero or near-zero cannabinoid expression. In some embodiments, the method can include germinating seeds from the hemp variety to obtain sprouts, wherein the sprouts have elevated levels of a cannflavin, compared to unsprouted hemp seeds or hemp seeds that lack the zero or near-zero cannabinoid phenotype. In some embodiments, the method can include adding at least one additional compound derived from Cannabis to the sprouts or product derived from the sprouts. In some embodiments, the method can include making a nutritional product from the sprouts, wherein the nutritional product has synergistic effects.

In some embodiments, the cannflavin can be CFA and/or CFB.

In some embodiments, the zero or near-zero cannabinoid expression can include expression of less than 0.1% cannabinoids by weight in the mature flower.

In some embodiments, the sprouts can include at least 0.0002% of cannflavin by weight.

In some embodiments, the at least one additional compound derived from Cannabis can include one or more of a non-THC cannabinoid a terpenoid, and any combination thereof.

In some embodiments, the at least one additional compound can include seed hulls of a Cannabis plant or a compound derived from said seed hulls. In some embodiments, the at least one additional compound can be cannabisin B or caffeoyltyramine.

In some embodiments, the one additional compound can include leaves or roots of a Cannabis plant or a compound derived from said leaves or roots. In some embodiments, the one additional compound can be canniprene or friedelin.

In some embodiments, the one additional compound can be caryophyllene or cannabigerol.

In some embodiments, the synergistic effects can be either or both of anti-inflammatory activity and anti-oxidant activity.

In some embodiments, the product is in the form of fresh sprouts, lyophilized sprouts, a powder, meal, capsule, bar, or the like.

Some embodiments of the invention relate to a nutritional product produced by the methods provided herein. In some embodiments, the nutritional product can be in a bulk form suitable for use as an ingredient in formulating one or more finished products.

Some embodiments of the invention relate to a finished product including the nutritional product.

Some embodiments of the invention relate to a method for treating a medical condition using the nutritional product. In some embodiments, the method can include providing the nutritional product to a subject and administering the nutritional product to a subject. In some embodiments, the subject is a human or an animal. In some embodiments, the medical condition is an inflammatory disease, cancer, a neurodegenerative disease, or the like.

### DETAILED DESCRIPTION

The present invention relates to hemp products, such as sprouted hemp seed products, and methods for producing the same. The methods preserve or improve the nutritional values of hemp seeds compared to unprocessed hemp seeds.

In some embodiments, the hemp seed product, such as a sprouted hemp seed product, can be a nutritional product produced in a form for consumption. The food form can be fresh sprouts, lyophilized sprouts, a powder, meal, capsule, bar, animal food, and/or any form that can be used as a nutritional supplement for humans or animals. The hemp seed product can have high levels of cannflavins. For example, sprouted hemp seed products can have up to 0.0008% cannflavin by weight depending on the chemovar. For example, the cannflavin concentration can be 0.0001%, 0.0002%, 0.0003%, 0.0004%, 0.0005%, 0.0006%, 0.0007%, or more.

In some embodiments, the sprouted hemp seed product demonstrates an inversely proportional concentration relationship to the eventual cannabinoid concentration of the variety's flower (absent or lowest in higher potency THC-chemovars and highest in zero cannabinoid chemovars, such as “Ermo”). For purposes of simplicity, the term “zero cannabinoids” is used in this application, however the term can be used to describe any variety that produces zero cannabinoids or near-zero cannabinoids such as below 0.1% cannabinoid by weight in mature flowers of that variety.

Thus, some embodiments of the invention relate to a method for making a nutritional product from hemp seeds. In some embodiments, the method can include providing a hemp variety with a zero or near-zero cannabinoid phenotype and germinating seeds from the hemp variety to obtain sprouts, wherein the sprouts have elevated levels of cannflavin compared to unsprouted hemp seeds or hemp seeds that lack the zero or near-zero cannabinoid phenotype. The cannflavin that is increased after sprouting can be Cannflavin A (CFA) and/or Cannflavin B (CFB), which are difficult to separate analytically. The nutritional hemp seed product can include both CFA and CFB.

While embodiments of the invention can include active ingredients, inert ingredients, flavors, and other formulation components, preferred embodiments begin with a primary blend. A primary blend is preferably a synergistic combination containing two or more active ingredients and, optionally, additional ingredients. The primary blends can then be combined with other ingredients to produce a final product. Accordingly, where concentrations, concentration ranges, or amounts, are given herein, such quantities can be in reference to a primary blend or blends. Thus, when a primary blend is further modified by addition of other ingredients to produce a formulation, the concentrations of the active ingredients are reduced proportional to the presence of other ingredients in the product. In some embodiments, the nutritional product can be capable of being used in combination with any food or beverage for human or animal consumption.

The hemp seed product can have otherwise similar or improved nutritional values as compared to unprocessed hemp seed. “Similar” can be defined as 50%, 60%, 70%, 80% or more, as measured by any single factor of comparison. Alternatively, “similar” can involve at least two factors of comparison wherein all factors on which the comparisons are based are within 50% or 60% or 70% or 80% or more. The nutritional value can be the concentrations or ratios of fatty acids, for example ratios of EFAs. The nutritional value can be the concentration of a polyphenol, flavonoid, or flavonol. The value can be of the primary blend and can be modified by the addition of other ingredients as described above.

The method can also include adding at least one additional compound derived from Cannabis to the sprouts or product derived from the sprouts; and/or making a nutritional product from the sprouts. In some embodiments the nutritional product has synergistic effects.

In some embodiments, the at least one additional compound is derived from a Cannabis part selected from leaves, seed hulls, roots, or the like. Further information on roots can be found in Ryz, N. R., D. J. Remillard, and E. B. Russo. 2017. “Cannabis Roots: A Traditional Therapy with Future Potential for Treating Inflammation and Pain.” Cannabis and Cannabinoid Research 2: 210-216. https://doi.org/10.1089/can.2017.0028, which is hereby fully incorporated by reference herein.

The at least one additional compound derived from Cannabis can be a non-THC cannabinoid, terpene, polyphenol, flavonoid, or flavonol. Each compound can make up between about 0.1% to about 99%, by weight, of the composition mixture. The Cannabis compounds, when combined, can have a synergistic effect. In some embodiments, the composition can include at least three, four, or five or more active compounds, in any effective combination. The compositions of the present invention can include any of the following Cannabis compounds listed below, or mixtures thereof:

TABLE 1
Exemplary Cannabinoids

| | |
|---|---|
| CBD (cannabidiol) | CBDA (cannabidiolic acid) |
| CBN (cannabinol) | CBG (cannabigerol) |
| CBC (cannabichromene) | CBL (cannabicyclol) |
| CBV (cannabivarin) | CBCV (cannabichromevarin) |
| CBDV (cannabidivarin) | CBGM (cannabigerol |
| | monomethyl ether) |
| CBGV (cannabigerovarin) | CBT (cannabicitran) |
| CBE (cannabielsoin) | CBGA (cannabigerolic acid) |
| CBCA (cannabichromenic acid) | THCV (tetrahydrocannabivarin |
| THCVA (tetrahydrocannabivarinic acid) | |

TABLE 2
Exemplary Terpenes

| | | | |
|---|---|---|---|
| 3-Carene | Alpha Pinene | Beta-caryophyllene | Beta-elemene |
| Beta-pinene | Bisabolol | Borneol | Camphene |
| Camphor | Caryophyllene | Caryophyllene Oxide | Cedrene |
| Cedrol | Delta 3 Carene | Eucalyptol | Fenchol |
| Fenchone | Geraniol | Geranyl Acetate | Guaiol |
| Humulene | Isoborneol | Isopulegol | Limonene |
| Linalool | Menthol | Myrcene | Nerol |
| Nerolidol | Ocimene | Phellandrene | Phytol |
| Sabinene | Terpinene | Terpineol | Terpinolene |
| Valencene | | | |

TABLE 3
Exemplary Flavonoids

| | | | |
|---|---|---|---|
| Cannflavin A | Cannflavin B | Cannflavin C | Orientin |
| Quercetin | Silymarin | Kaempferol | Apigenin |

TABLE 4
Exemplary Polyphenols

| | | |
|---|---|---|
| Cannabisin B | Caffeoyltyramine | Canniprene |

Further information on canniprene can be found in Allegrone, G., F. Pollastro, G. Magagnini, O. Taglialatela-Scafati, J. Seegers, A. Koeberle, O. Werz, and G. Appendino. 2017. “The bibenzyl canniprene inhibit the production of pro-inflammatory eicosanoids and selectively accumulates in some cannabis strains.” Journal of Natural Products 80 (3): 731-734. https://doi.org/10.1021/acs.jnatprod.6b01126, which is hereby fully incorporated by reference herein. Further information on Cannabis compounds can be found in Russo, E. B., and J. Marcu. 2017. “Cannabis pharmacology: The usual suspects and a few promising leads.” Advances in Pharmacology 80: 71-138. https://doi.org/10.1016/bs.apha.2017.03.004, which is hereby fully incorporated by reference herein.

“Compound derived from a Cannabis plant,” as used herein, can be defined as a compound naturally found in Cannabis . The actual compound used in the composition that is naturally found in Cannabis can be produced from another source.

In some embodiments, the sprouted hemp seed product can include a compound derived from seed hulls of a Cannabis plant. In some embodiments, the compound is a polyphenol compound such as cannabisin B or caffeoyltyramine (produced by hemp seed hulls). Both compounds have demonstrated antioxidant activity against DPPH radicals, as well as prominent inhibition of human LDL oxidation (Chen, T., He, J., Zhang, J., Li, X., Zhang, H., Hao, J., & Li, L. (2012). The isolation and identification of two compounds with predominant radical scavenging activity in hempseed (seed of Cannabis sativa L.). Food Chem, 134(2), 1030-1037. doi:10.1016/j.foodchem.2012.03.009). In subsequent work (Chen, T., Hao, J., He, J., Zhang, J., Li, Y., Liu, R., & Li, L. (2013). Cannabisin B induces autophagic cell death by inhibiting the AKT/mTOR pathway and S phase cell cycle arrest in HepG2 cells. Food Chem, 138(2-3), 1034-1041. doi:10.1016/j.foodchem.2012.11.102), cannabisin B produced antiproliferative effects in HepG2 human hepatic carcinoma cells (dose-dependently up to 500 μM). All of the foregoing references are fully incorporated by reference herein.

In some embodiments, the product (primary blend) or formulation including the product can include about, 0.1%, 1%, 5%, 10%, or more cannabinoid by weight.

In some embodiments, the product or formulation including the product can include about 0.01%, 0.1%, 1%, 5% or more terpenoid or terpene by weight.

In some embodiments, the product or product including the composition can include about 0.0001%, 0.001%, 0.01%, 0.1%, 1%, 5%, or more flavonoid by weight.

In some embodiments, the product or formulation including the product can include about 0.0001%, 0.001%, 0.01%, 0.1%, 1%, 5%, or more polyphenol by weight.

The two compounds together can have synergistic effects as provided in Table 5, wherein the ingredients are referred to as a primary ingredient and a secondary ingredient. In some embodiments, the composition includes a cannflavin plus cannabisin B (from seed coat) (Formulas 121 or 122). In some embodiments, the composition includes a cannflavin plus canniprene (from leaves) (Formulas 125 or 126). In some embodiments, the composition includes a cannflavin plus friedelin (from roots) (Formulas 127 or 128). In some embodiments, the composition can include a cannflavin plus beta-caryophyllene (from flower) (Formulas 39 or 40). In some embodiments, the composition includes a cannflavin plus cannabigerol (from flower) (Formulas 7 or 8).

TABLE 5
Synergistic pairwise combinations

Primary

| Secondary | Cannflavin A | Cannflavin B |
|---|---|---|
| CBD | 1 | 2 |
| CBDA | 3 | 4 |
| CBN | 5 | 6 |
| CBG | 7 | 8 |
| CBC | 9 | 10 |
| CBL | 11 | 12 |
| CBV | 13 | 14 |
| CBCV | 15 | 16 |
| CBDV | 17 | 18 |
| CBGM | 19 | 20 |
| CBGV | 21 | 22 |
| CBT | 23 | 24 |
| CBE | 25 | 26 |
| CBGA | 27 | 28 |
| CBCA | 29 | 30 |
| THCV | 31 | 32 |
| THCVA | 33 | 34 |
| 3-Carene | 35 | 36 |
| Alpha Pinene | 37 | 38 |
| Beta-caryophyllene | 39 | 40 |
| Beta-elemene | 41 | 42 |
| Beta-pinene | 43 | 44 |
| Bisabolol | 45 | 46 |
| Borneol | 47 | 48 |
| Camphene | 49 | 50 |
| Camphor | 51 | 52 |
| Caryophyllene | 53 | 54 |
| Caryophyllene | 55 | 56 |
| Oxide | | |
| Cedrene | 57 | 58 |
| Cedrol | 59 | 60 |
| Delta 3 Carene | 61 | 62 |
| Eucalyptol | 63 | 64 |
| Fenchol | 65 | 66 |
| Fenchone | 67 | 68 |
| Geraniol | 69 | 70 |
| Geranyl Acetate | 71 | 72 |
| Guaiol | 73 | 74 |
| Humulene | 75 | 76 |
| Isoborneol | 77 | 78 |
| Isopulegol | 79 | 80 |
| Limonene | 81 | 82 |
| Linalool | 83 | 84 |
| Menthol | 85 | 86 |
| Myrcene | 87 | 88 |
| Nerol | 89 | 90 |
| Nerolidol | 91 | 92 |
| Ocimene | 93 | 94 |
| Phellandrene | 95 | 96 |
| Phytol | 97 | 98 |
| Sabinene | 99 | 100 |
| Terpinene | 101 | 102 |
| Terpineol | 103 | 104 |
| Terpinolene | 105 | 106 |
| Valencene | 107 | 108 |
| Cannflavin C | 109 | 110 |
| Orientin | 111 | 112 |
| Quercetin | 113 | 114 |
| Silymarin | 115 | 116 |
| Kaempferol | 117 | 118 |
| Apigenin | 119 | 120 |
| Cannabisin B | 121 | 122 |
| Caffeoyltyramine | 123 | 124 |
| Canniprene | 125 | 126 |
| Friedelin | 127 | 128 |

The ratio of the synergistic ingredients can range from 10:1 to 1:5000 or less. For example, the ratio can be about 10:1, 1:1, 1:10, 1:500, 1:1000, 1:1500, 1:2000, 1:2500, 1:3000, 1:3500, 1:4000, 1:4500, 1:5000 depending on the synergistic ingredients. For example, cannflavins 0.001% plus cannabisin B 0.01% (from seed coat) is a 1:10 ratio; cannflavins 0.001% plus canniprene 0.0001% (from leaves) is a 10:1 ratio; cannflavins 0.001% plus friedelin 1% (alkaloid from roots) is a 1:1000 ratio; cannflavins 0.001% plus beta-caryophyllene 2% (from flower) is a 1:2000 ratio; and cannflavins 0.001% plus cannabigerol 5% (from flower) is a 1:5000 ratio. However, it is within the scope of the invention to provide formulations in which synergistic ratios are adapted to particular uses.

Table 6 lists major ratios of pairwise combinations of synergistic ingredients of the invention, named as ratios A through S. To make these ratios workable for the purpose of the table, the “1 part” cannflavin can be defined as 5 micrograms (0.0005%, 5micrograms/gram) where the “1 part” secondary ingredient can be defined as 1 gm. However, it should be noted that ratios are all expressed as amounts relative to each other and any units of mass can be substituted for grams so long as the proportionality remains the same. While the table in this figure provides a range of ratios that can be useful, it is within the scope of the invention to provide formulations in which synergistic ratios are adapted to particular uses.

TABLE 6
Exemplary Ratios

| | Secondary | | | | | |
|---|---|---|---|---|---|---|
| Cannflavin | 1 part S2 | 2 parts S2 | 3 parts S2 | 6 parts S2 | 9 parts S2 | 12 parts S2 |
| 1 part S1 | 1:1 (A) | 1:2 (G) | 1:3 (J) | 1:6 (M) | 1:9 (N) | 1:12 (Q) |
| 2 parts S1 | 2:1 (B) | 2:2 (A) | 2:3 (K) | 2:6 (J) | 2:9 (O) | 2:12 (M) |
| 3 parts S1 | 3:1 (C) | 3:2 (H) | 3:3 (A) | 3:6 (G) | 3:9 (J) | 3:12 (R) |
| 6 parts S1 | 6:1 (D) | 6:2 (C) | 6:3 (B) | 6:6 (A) | 6:9 (K) | 6:12 (G) |
| 9 parts S1 | 9:1 (E) | 9:2 (I) | 9:3 (C) | 9:6 (H) | 9:9 (A) | 9:12 (S) |
| 12 parts S1 | 12:1 (F) | 12:2 (D) | 12:3 (L) | 12:6 (B) | 12:9 (P) | 12:12 (A) |

Likewise, the synergistic formulations of the invention can include additional ingredients to further synergistically enhance the efficacy of a pairwise combination. In such embodiments, a paired combination is enhanced with a tertiary ingredient selected from Table 7. In some embodiments, a quaternary ingredient can also be selected from Table 7. Likewise in other embodiments, additional synergists can be selected, either from Table 7 or from other sources based upon desired effects.

TABLE 7
Exemplary tertiary and quaternary synergistic ingredients

| | code |
|---|---|
| THCV | aa |
| THCVA | ab |
| CBD | ac |
| CBDA | ad |
| CBN | ae |
| CBG | af |
| CBC | ag |
| CBL | ah |
| CBV | ai |
| CBCV | aj |
| CBDV | ak |
| CBGM | al |
| CBGV | am |
| CBT | an |
| CBE | ao |
| CBGA | ap |
| CBCA | aq |
| Cannflavin A | ar |
| Cannflavin B | as |
| Cannflavin C | at |
| Orientin | au |
| Quercetin | av |
| Silymarin | aw |
| Kaempferol | ax |
| Cannabisin B | ay |
| Caffeoyltyramine | az |
| Canniprene | ba |
| 3-Carene | bb |
| Alpha Pinene | bc |
| Beta Caryophyllene | bd |
| Beta-elemene | be |
| Beta Pinene | bf |
| Bisabolol | bg |
| Borneol | bh |
| Camphene | bi |
| Camphor | bj |
| Caryophyllene | bk |
| Caryophyllene Oxide | bl |
| Cedrene | bm |
| Cedrol | bn |
| Delta 3 Carene | bo |
| Eucalyptol | bp |
| Fenchol | bq |
| Fenchone | br |
| Geraniol | bs |
| Geranyl Acetate | bt |
| Guaiol | bu |
| Humulene | bv |
| Isoborneol | bw |
| Isopulegol | bx |
| Limonene | by |
| Linalool | bz |
| Menthol | ca |
| Myrcene | cb |
| Nerol | cc |
| Nerolidol | cd |
| Ocimene | ce |
| Phellandrene | cf |
| Phytol | cg |
| Sabinene | ch |
| Terpinene | ci |
| Terpineol | cj |
| Terpinolene | ck |
| Valencene | cl |
| Friedelin | cj |

The terms “synergistic” and “synergistically effective” are used in the present invention to mean a biological effect created from the application of two or more agents that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15(1): 20-23, 1967; the entire contents of the foregoing is fully incorporated by reference herein.

In some embodiments, the hemp seed product can have similar or improved antioxidant activity compared to unprocessed seeds. Antioxidant activity can be quantified by oxygen radical absorbance capacity (ORAC), 2,2-diphenyl-1-picrylhydrazyl (DPPH) radical scavenging activity, cellular antioxidant activity in red blood cells (CAA-RBC) and hemolysis tests. In some embodiments, the hemp seed product can have similar or improved anti-mutagenic effects compared to unprocessed seeds.

Germination

The methods can include sprouting or germination. The terms “sprouting” and “germination” can be used interchangeably. Germination is an effective method for improving food quality and increasing food nutritive value. Sprouting can reduce or eliminate toxic, anti-metabolic factors (e.g., as occurs in the breaking down of genistein in soybeans) which can arise during processing. Sprouting can likewise increase concentrations of beneficial phytochemicals (e.g., as is the case with sulforaphane in broccoli sprouts).

In some embodiments, the method can include germinating hemp seeds in a seed germinator for an amount of time. The amount of time can be up to 3, 4, or 5 days. A non-limiting example of a seed germinator suitable for use in the invention is VitaSeed. In some embodiments, the resulting sprouts can be dried into an extract.

The general methodology for sprouting hemp seeds according to the invention is provided. Specific examples follow, which incorporate particular time periods, temperatures, etc. However, for purposes of the invention, the general methodology can be adapted with minor variations, as would be apparent to those of skill in the art.

The general methodology covers all aspects of sprout or hemp seed sprout production, at every stage from seed selection to packaging to the formation of baked goods. Of course, not all aspects of the general methodology are required to fall within the scope of the invention. Those steps of the general methodology which are optional to the invention are identified as such.

The method steps can include culling the seeds for foreign objects, cleaning the seeds, and germinating the seeds.

Seed selection is initially conducted as an optional aspect of the invention. Of course, any type of seed that sprouts can be used. For seed selection, one optional criterion is that the seed be “organic,” as popularly defined to mean pesticide-free. The seed is optimally of uniform density, unbroken, non-discolored, mold-free, and should have a germination rate of approximately 98%, as determined in advance of conducting the sprouting methodology. When other types of seed are selected either for use alone or in combination with hemp seed, similar criteria can be applied. Although use of seeds with a high germination rate is beneficial, some seed varieties do not have a 98% germination rate but may still have desirable properties when sprouted. Accordingly, the use of such seeds, and sprouts of such seeds are still within the scope of the invention.

Seed hydration can be conducted in a variety of different ways. The general case can be varied, depending on the conditions under which the sprouting is to occur. The process outlined herein can be optimal for hemp seed and hemp seed mixtures, but need not be followed stringently. The sprouting process can follow conventional sprouting methodologies.

Initially, the selected seed is spread out in an appropriate thickness. Then, water is added to begin the sprouting process. In some embodiments, the water can be added to the seed in a plurality of separate additions. In some embodiments, the seeds are washed over exemplary time periods. For example, in some embodiments, seeds are washed 1-5 times or more a day.

In some embodiments, water additions can be made periodically, for example every hour or every two hours, followed by agitation for a period of time adequate to distribute the water throughout the seeds. In some embodiments, agitation can be done at a relatively slow rate, such as from 5 to 100 rpm. The agitation period can be from about 2 minutes to about 15 minutes, for example, for 10 minutes after each subsequent addition of water.

Of course, any variation of this process which allows for subsequent additions of water to be added at periodic intervals with mixing is encompassed.

The sprouting is allowed to continue for a period of time ranging from until the sprout emerges from the seed to a period of time in which the sprout is appropriately grown, as described further below.

Hemp seed sprouts and hemp seed blends can be used as fresh sprouts, as they have enhanced nutritional characteristics compared to unsprouted hemp seeds (for example, enhanced Omega-3 fatty acid content), and increased stability and shelf life as compared to unprocessed hemp seeds.

In some embodiments, the hemp seed sprouts formed according to the invention are sprouted for a period of time between about ¼ of a day (6 hours) and about 7 days. For example, a sprouting period ranging from 1 to 5 days is an exemplary time period. In some embodiments, the sprouting period is a minimum of 2 days. The sprouts can be used with the invention any time after the sprout begins to emerge.

As an alternative to consumption of fresh sprouts or fresh sprout blends, the hemp seed sprouts, blends or other types of sprouted seed can be dried according to an optional aspect of the invention, as described herein.

In some embodiments, the sprouts are lyophilized (freeze-dried). In some embodiments, the dried hemp sprouts can be ground in a low-speed grinder. Suitable methods for lyophilization and grinding of the products can readily be determined by those skilled in the art.

Grinding can be followed by sieving to produce a uniform hemp sprout powder with about 0.5% moisture. This hemp sprout powder can be used as a nutraceutically stable food product in itself. Alternatively, the powder obtained can be dry steam distilled to obtain oils and liquid extracts. The sprout powder obtained can alternatively be encapsulated, for example with an enteric coating.

In some embodiments, a milling process can occur. Milling can be done with any type of mill, for example with a Fritz mill, which has a series of rotating blades through which sprouts pass via gravity. Optionally, the sprouts are ground to a coarse granule size, for optimal incorporation into baked products. The milled product can be referred to herein as a “powder” regardless of whether the product is a coarse grind or fine grind. Either coarse or fine grinds fall within the scope of the invention.

If an extract of the product is desired, the dried or fresh sprouts can be mixed in water, or an oil, and centrifuged. An aqueous supernatant thus obtained can be lyophilized. The freeze-dried extract can be added to solid foods to obtain nutraceutically enriched solid food products. In addition, the supernatant can also be used to obtain nutritionally enriched beverages. In order to obtain nutraceutically enriched beverages, the supernatant of the aqueous extract can be acidified with 1 ml of dilute citric acid solution. The solution thus obtained, rich in nutraceuticals, can be added to suitable vegetable or fruit base to obtain the desired nutraceutically enriched beverage.

The packaging of the final product can be of any acceptable type, for example, a Ziplock™ or other type of self-sealing bag, which can be enclosed in a type of packaging acceptable to a consumer. An exterior cotton bag sized to fit an inner plastic bag can be used. Optionally, product information can be attached to such an exterior bag. The milled products so formed can be used to increase the nutritional content (for example, Omega-3 fatty acids and dietary fiber) of baked products, such as breads, muffins, and cooked cereals. Further, these milled products can be added to liquid or semi-solid products such as juices or yogurt. The milled hemp seed product formed in this way has a shelf life of over 1 year, and the nutritional value of the product can supplement many foods that are not high in such components as Omega-3 fatty acids and dietary fiber. A number of other nutritional components are enhanced in hemp seed sprouts as compared to unprocessed hemp seeds.

Powder Product

The sprouts formed according to the invention can be blended with other foods, such as processed foods not known to or expected to contain CFA or CFB. In this way, powders containing the sprouts or extracts derived from the sprouts can be used as nutraceutical components to supplement foods.

In some embodiments, the powder contains a cannflavin and is capable of delivering the cannflavin to a human or animal upon consumption.

Methods of Treatment

Some embodiments relate to methods of treating or preventing a medical condition using the hemp seed product. The medical condition can be, for example, an inflammatory disease (e.g., the inflammatory bowel diseases: Crohn's disease and ulcerative colitis), cancer, Alzheimer disease, or the like. Non-limiting examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disorder (IBD) (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections, and parasitic infections also can have immune and/or inflammatory components and/or etiology.

In some embodiments, the method includes administering the hemp seed product in an amount that equates to delivering about 50, 60, 70, 80, 90, 100, 110, 120, 140 μg/day of cannflavin. For example, the hemp seed product can be fresh hemp seed sprouts which are administered at 2, 3, 4, 5, 20, or more grams a day. Effective amounts of the cannflavin molecules described herein can vary according to factors such as the disease state, age, sex, and weight of the subject. Dosage or treatment regimens can be adjusted to provide the optimum therapeutic response, as is understood by a skilled person.

Further information can be found in Frassinetti S et al. 2018. Nutraceutical potential of hemp (Cannabis sativa L.) seeds and sprouts. Food Chem 262:56-66 and Werz O et al. 2014. Cannflavins from hemp sprouts, a novel cannabinoid-free hemp food product, target microsomal prostaglandin E2 synthase-1 and 5-lipoxygenase. PharmaNutrition vol. 2, issue, pages 53-6, which are both fully incorporated by reference herein.

### EXAMPLES

### Example 1

The following example illustrates a method for processing hemp seeds.

Seeds were germinated in the dark for 5 days in a VitaSeed. Multiple varieties were used, including Ermo, an extremely low cannabinoid chemovar, developed in Rovigo, Italy, at 45° North latitude. The processed Ermo demonstrated a 30% increase of GLA (from 1.08 to 1.42%) after sprouting. Ermo produced especially high titers of cannflavin with no phytocannabinoids. The yield from 900 g of dried material was 95 mg cannflavin A (0.011%). The lipid content decreased from 36% in seeds to 31% in sprouts with EFA ratios preserved.

TABLE 8
Fatty acids composition of seeds and sprouts from
the Ermo variety of hemp (each number represents
the mean of three replicates ± standard deviation).

| Fatty Acid (Relative %) | Seeds | Sprouts |
|---|---|---|
| Linoleic | 57.53 ± 0.2 | 58.00 ± 0.31 |
| α-Linolenic | 24.53 ± 0.14 | 24.55 ± 0.17 |
| Oleic | 9.97 ± 0.06 | 10.01 ± 0.07 |
| γ-Linolenic | 1.08 ± 0.01 | 1.42 ± 0.03 |
| Palmitic | 4.96 ± 0.13 | 4.18 ± 0.207 |
| Stearic | 1.94 ± 0.01 | 1.85 ± 0.04 |

O. Werz et al. PharmaNutrition 2 (2014) 53-60

### Example 2

The following example illustrates the effects of CFA.

Cannflavin A (CFA) suppressed PGE2, the primary mediator of inflammation. CFA directly inhibited mPGES-1, a target in inflammation and cancer. CFA did not significantly inhibit COX-1 (Non-steroidal anti-inflammatory drugs (NSAIDs) and drugs that do cause gastritis and ulcers). CFA did not significantly inhibit COX-2 (drugs whose adverse events include myocardial infarction and cerebrovascular accidents).

TABLE 9
Effects of CFA (1) on various eicosanoid-forming enzymes and cellular functions
of neutrophils and monocytes. CFA (1) or reference inhibitors (at the indicated
concentrations) were added to the respective enzymes or freshly isolated human
blood cells 15 min prior induction of the enzyme reaction. Data (means ± S.E.,
n = 3-5) are expressed as IC₅₀ values and as percentage of the inhibition
of the enzyme activity vs. the uninhibited vehicle (0.1% DMSO) control.

| | CFA(1) IC₅₀ [μm]; % | Reference control, % |
|---|---|---|
| Enzyme/assay | inhibition at 10 μM | inhibition at indic. conc. |
| COX-1, platelets | >10 μM; 26 ± 2% | Indomethacin (20 μM), 95 ± 5% |
| COX-1, cell-free | >10 μM; 36 ± 2% | Indomethacin (20 μM), 81 ± 8% |
| COX-2, monocytes | 8.8 μM; 56 ± 8% | Celecoxib (5 μM), 80 ± 5% |
| COX-2, cell-free | >10 μM; 35 ± 12% | Celecoxib (5 μM), 78 ± 8% |
| cPLA₂, cell-free | >10 μM; 26 ± 8% | Pyrrolidine-1 (5 μM), 79 ± 3% |
| mPGES-1, cell-free | 1.8 μM; 90 ± 1% | MK-886 (10 μM), 83 ± 1% |
| 5-LO cell-free | 0.9 μM; 88 ± 3% | Zileuton (3 μM), 80 ± 4% |
| 5-lo, PMNL, A23187 | 2.4 μM; 98 ± 2% | BWA4C (0.3 μM), 99 ± 2% |
| 5-lo, PMNL, A23187 + AA | 1.6 μM; 91 ± 1% | BWA4C (0.3 μM), 76 ± 2% |
| DPPH assay | No effect | Ascorbate (50 μM), 80 ± 6% |
| Cell viability | no effect | staurosporine (3 μM), 80 ± 5% |

(Werz, O., J. Seegers, A. M. Schaible, C. Weinigel, D. Barz, A. Koeberle, G. Allegrone, F. Pollastro, L Zampieri, G. Grassi, and G. Appendino. 2014. “Cannflavins from hemp sprouts, a novel cannabinoid-free hemp food product, target microsomal prostaglanin E2 synthase-1 and 5 lipoxygenase.” PharmaNutrition 2: 53-60.)

CFA directly inhibited mPGES-1, a major target in development of drugs to treat inflammation and inflammatory-induced forms of cancer.

CFA potently inhibited 5-LO activity at IC50 of 0.9 μM. In neutrophils, 5-LO was suppressed at IC50 of 1.6 μM.

Unlike some flavonoids, cannflavin A did not demonstrate innate antioxidant activity. Of note, cannflavin A produced no evidence of cytotoxicity and no deleterious effects on human cell viability.

### Example 3

A hemp seed product in the form of hemp sprout meal is made from lyophilized hemp sprouts, including hulls. The product has full hemp seed nutrition plus the appropriate daily dosage of cannflavins, cannabisin B, and caffeoyltyramine. The product is sold in 1 kg tubs with recommended dosing of 2 grams a day with meals (e.g., mixed in shake, or cereal). Daily users of the product enjoy benefits including the advantages of high-quality protein and essential fatty acid intake; anti-inflammatory benefits; and decreased risk of autoimmune conditions, Alzheimer's disease, and the like.

### Example 4

A hemp seed product is made as described in Example 3 and is packaged for use in the form of hemp sprout capsules. The product is sold in 500 mg capsules with recommended dosing of two 500 mg capsules twice a day with meals. Daily users of the product enjoy benefits including advantages of high-quality protein and essential fatty acid intake; anti-inflammatory benefits; and decreased risk of autoimmune conditions and Alzheimer' s disease, and the like.

### Example 5

A hemp seed product is prepared in the form of a food bar. The food bar can include sprouted hemp with cannflavin, combined with, for example, apricots, almond sprouts, pistachios, pomegranate seeds, goji berries, and the like. The food bar can include sprouted hemp with cannflavin, amaranth, pepita (pumpkinseed) sprouts, wild blueberries. Daily users of the product enjoy benefits including advantages of high-quality protein and essential fatty acid intake; anti-inflammatory benefits; and decreased risk of autoimmune conditions and Alzheimer's disease, and the like.

### Example 6

Sprouted hemp seed powder is formed, and is included in a conventional recipe for a baked good. In the recipe, the sprouted hemp seed powder is included as a substitute for 20% of the flour content of the recipe on a 1:1 volume basis.

The product containing the sprouted hemp seed powder show increased content of fatty acids, and CFA. The products that are formed with the sprouted hemp seed powder are of acceptable quality, and are reported to be very palatable by tasting panels. Further, the products containing the hemp seed powder show increased shelf life and maintain the moisture content better than the conventional baked good product.

### Example 7

A hemp seed product including a cannflavin plus cannabisin B (from seed coat) (Formulas 121 or 122) is prepared and found to have synergistic effects.

### Example 8

A hemp seed product including a cannflavin plus canniprene (from leaves) (Formulas 125 or 126) is prepared and found to have synergistic effects.

### Example 9

A hemp seed product including a cannflavin plus friedelin (from roots) (Formulas 127 or 128) is prepared and found to have synergistic effects.

### Example 10

A hemp seed product including a cannflavin plus beta-caryophyllene (from flower) (Formulas 39 or 40) is prepared and found to have synergistic effects.

### Example 11

A hemp seed product including a cannflavin plus cannabigerol (from flower) (Formulas 7 or 8) is prepared and found to have synergistic effects.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature, or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of making a nutritional product comprising:
a) providing a hemp variety with a zero or near-zero cannabinoid expression;
b) germinating seeds from the hemp variety to obtain sprouts, wherein the sprouts have elevated levels of a cannflavin, compared to unsprouted hemp seeds or hemp seeds that lack the zero or near-zero cannabinoid phenotype;
c) adding at least one additional compound derived from Cannabis to the sprouts or product derived from the sprouts; and
d) making a nutritional product from the sprouts; wherein the nutritional product has synergistic effects.

2. The method of claim 1, wherein the cannflavin is CFA and/or CFB.

3. The method of claim 1, wherein the zero or near-zero cannabinoid expression comprises expression of less than 0.1% cannabinoids by weight in the mature flower.

4. The method of claim 1, wherein the sprouts comprise at least 0.0002% of cannflavin by weight.

5. The method of claim 1, wherein the at least one additional compound derived from Cannabis comprises one or more of a non-THC cannabinoid a terpenoid, and any combination thereof.

6. The method of claim 1, wherein the at least one additional compound comprises seed hulls of a Cannabis plant or a compound derived from said seed hulls.

7. The method of claim 6, wherein the at least one additional compound is cannabisin B or caffeoyltyramine.

8. The method of claim 1, wherein the one additional compound comprises leaves or roots of a Cannabis plant or a compound derived from said leaves or roots.

9. The method of claim 8, wherein the one additional compound is canniprene or friedelin.

10. The method of claim 1, wherein the one additional compound is caryophyllene or cannabigerol.

11. The method of claim 1, wherein the synergistic effects comprise either or both of anti-inflammatory activity and anti-oxidant activity.

12. The method of claim 1, wherein the product is in the form of fresh sprouts, lyophilized sprouts, a powder, meal, capsule, or bar.

13. A nutritional product produced by the method of claim 1.

14. The nutritional product of claim 12, in a bulk form suitable for use as an ingredient in formulating one or more finished products.

15. (canceled)

16. A method for treating a medical condition using the nutritional product of claim 13, comprising: providing the nutritional product to a subject and administering the nutritional product to a subject.

17. The method of claim 16, wherein the subject is a human or an animal.

18. The method of claim 16, wherein the medical condition is an inflammatory disease, cancer, or a neurodegenerative disease.
